(19) United States
(12) Patent Application Publication
(10) Pub. No.: US 2026/0086527 A1
(43) Pub. Date: Mar. 26, 2026

(54) LIVING-PHYSICS MEASUREMENT AND CONTROL SYSTEM FOR ENERGETIC ORGANIZATION IN OPEN SYSTEMS

(71) Applicant: Hila Shimshi, San Anselmo, CA (US)

(72) Inventor: Hila Shimshi, San Anselmo, CA (US)

(21) Appl. No.: 19/396,253

(22) Filed: Nov. 20, 2025

Related U.S. Application Data
(60) Provisional application No. 63/724,125, filed on Nov. 22, 2024.
(60) Provisional application No. 63/810,638, filed on May 22, 2025.

(51) Int. Cl. G05B 19/05; G01N 33/483

(52) U.S. Cl. CPC G05B 19/058; G01N 33/4833; G05B 19/054; G05B 2219/1133; G05B 2219/15087; G05B 2219/2639

(57) ABSTRACT

A living-physics measurement and control system integrates a sensor array, analog front-end, controller module, and feedback actuator network to enable real-time quantification and modulation of energetic organization in open systems. The sensor array comprises thermal, mechanical/vibration, electrical/ionic, and optical sensors, providing multimodal data to the firmware and signal-processing unit. The controller module executes a coherence and entropy feedback control method, which includes system calibration, acquisition of synchronized sensor data, computation of order metrics such as coherence, entropy production, and information flux, and evaluation of energetic organization state. The feedback actuator network, including thermal actuators, mechanical actuators, ionic pumps, and optical emitters, receives actuator command set to dynamically adjust energy flow. The system generates quantitative metrics linking coherence, entropy, and information flow, enabling adaptive feedback to preserve or optimize functional order. The invention addresses the lack of integrated instrumentation and feedback for self-organizing, regenerative stability in open systems.

### CROSS REFERENCE TO RELATED APPLICATIONS

This utility patent application claims the priority dates and benefits of patent applications: 63/810,638 filed on 22 May 2025 and 63/724,125 filed on 22 Nov. 2024 The contents of which are incorporated by reference herein as if restated in this patent application.

### FIELD OF INNOVATION

The present invention relates to the measurement, quantification, and control of energetic organization within open systems. More particularly, it concerns methods and devices that monitor coherence, entropy production, and information flow across physical, chemical, or biological media to maintain functional order and regenerative stability. The invention bridges thermodynamics, information theory, and biophysics into an engineering framework for adaptive, self-organizing materials and sensors.

The present invention further provides a foundation for a class of regenerative vascular materials and associated control systems capable of directing, stabilizing, or restoring plant-derived or plant-mimetic tissues through coordinated electrical, mechanical, thermal, optical, and biochemical stimuli. Bio-derived vesicular sensing elements are delivered in free form or immobilized formats to modulate the differentiation environment, while multimodal sensors (impedance, ionic, optical, thermal, and mechanical) compute order-disorder metrics that drive closed-loop actuation. The methods enable xylem, phloem, hybrid, or novel vascular architectures to form within natural or engineered wood-like substrates.

### BACKGROUND

Non-equilibrium thermodynamics and biophysical control engineering have historically faced significant limitations in the development of integrated real-time instrumentation capable of measuring energetic organization within open systems. Conventional approaches have often relied on indirect or static measurements, which fail to capture the dynamic and transient nature of energy flows and organizational states in living or complex engineered systems. As a result, prior instrumentation has been unable to provide comprehensive, real-time insights into the evolving energetic landscape of open systems.

A further challenge in the field has been the absence of quantitative metrics that directly link coherence, entropy production, and information flow. Existing methodologies typically address these parameters in isolation, utilizing separate theoretical frameworks and measurement techniques. This compartmentalization has hindered the ability to establish unified, quantitative relationships among these fundamental aspects of non-equilibrium systems, thereby limiting the capacity to assess or predict system behavior in a holistic manner.

Another deficiency in prior art is the lack of feedback mechanisms designed to dynamically modulate energy flow in order to preserve functional order within open systems. Traditional control strategies in biophysical engineering have generally been static or pre-programmed, lacking the adaptability required to respond to fluctuating environmental conditions or internal system changes. Consequently, these approaches have been unable to maintain or restore order in the face of ongoing perturbations, leading to a loss of functional stability over time.

The field has also been constrained by the dominance of closed-system design principles, which typically regard disorder and entropy production as unavoidable waste. Such paradigms have prioritized the minimization of energy loss without considering the potential for harnessing or redirecting entropy production to sustain or enhance system organization. This perspective has limited the exploration of open-system dynamics, where continuous energy and matter exchange with the environment can be leveraged for maintaining order and function.

Finally, there has been a persistent inability to engineer adaptive, self-organizing materials that exhibit regenerative stability. Prior attempts at material design have largely focused on static or passively stable structures, lacking the capacity for active adaptation or self-repair in response to environmental changes or internal degradation. As a result, materials and systems developed under these paradigms have not demonstrated the regenerative or self-organizing properties observed in natural systems, restricting their applicability in dynamic or unpredictable environments.

### SUMMARY

The living-physics measurement and control system 100, in conjunction with the coherence and entropy feedback control method, provides an integrated platform for real-time measurement, analysis, and active regulation of energetic organization in open systems. The living-physics measurement and control system 100 comprises a sensor array 105, including thermal sensors, mechanical/vibration sensors, electrical/ionic sensors, and optical sensors 123, interfaced with an analog front-end, a controller module 106, a firmware and signal-processing unit, a power management subsystem, a clock and synchronization circuit, a feedback actuator network 107, a data storage and communication interface, and a power supply. The coherence and entropy feedback control method orchestrates system initialization, multimodal data acquisition, computation of order metrics, energetic organization assessment, and closed-loop feedback actuation to maintain or optimize system order. The living-physics measurement and control system 100 and the coherence and entropy feedback control method together enable adaptive, self-organizing, and regenerative control of complex material or biological systems.

The living-physics measurement and control system 100 addresses the lack of integrated real-time instrumentation for measuring energetic organization in open systems by providing a unified hardware and software architecture capable of simultaneous multimodal sensing, synchronized data acquisition, and real-time processing. The sensor array 105's combination of thermal, mechanical, electrical, and optical modalities, together with the analog front-end and firmware and signal-processing unit, enables comprehensive and time-aligned measurement of diverse physical parameters. This integration surpasses prior art, which typically relies on isolated or sequential measurements, by delivering continuous, high-resolution, and synchronized data streams that reflect the dynamic energetic organization of open systems.

The coherence and entropy feedback control method solves the absence of quantitative metrics linking coherence, entropy production, and information flow by implementing a computational pipeline that calculates an averaged coherence index, an entropy production rate, and an information flux metric from the multimodal sensor data stream. The firmware and signal-processing unit computes these order metrics in real time, and further derives a composite coherence-per-joule metric to quantify the efficiency of energetic organization. This approach provides a direct, quantitative linkage between physical order, thermodynamic dissipation, and information transfer, which is not available in conventional instrumentation that lacks such integrative metrics.

The feedback actuator network 107, coordinated by the controller module 106 and the coherence and entropy feedback control method, addresses the absence of feedback mechanisms to dynamically modulate energy flow and preserve functional order. The feedback actuator network 107 includes thermal actuators 172, mechanical actuators 154, ionic pumps 152, and optical emitters, which receive actuator command set determined by real-time assessment of energetic organization. The controller module 106 evaluates threshold violations and energetic organization assessment, then determines and applies actuator adjustments to restore or enhance system order. This closed-loop feedback capability enables dynamic, adaptive modulation of energy flows, in contrast to prior art systems that operate in open-loop or static configurations.

The living-physics measurement and control system 100 overcomes the dominance of closed-system design principles that treat disorder as inevitable waste by enabling open-system operation with active management of order and dissipation. The coherence and entropy feedback control method continuously updates baseline or learning parameters, allowing the system to adapt to changing environmental conditions and internal states. The system's ability to detect threshold violation and execute corrective feedback distinguishes it from traditional designs, which lack mechanisms for ongoing adaptation and typically accept entropy production as an unavoidable loss.

The living-physics measurement and control system 100 and the coherence and entropy feedback control method enable the engineering of adaptive, self-organizing materials with regenerative stability by providing a platform for continuous monitoring, assessment, and regulation of energetic organization. The distributed multi-node mesh variant extends these capabilities to spatially distributed systems, supporting collective adaptation and resilience. The system's capacity to maintain or restore optimized energetic organization states through real-time feedback and learning mechanisms represents a significant improvement over prior art, which is limited to static or externally controlled systems without intrinsic regenerative stability.

### BRIEF DESCRIPTION OF FIGURES

FIG. 1 System Overview: biophysical-biological control system at a high level.

FIG. 2 Tissue Chamber Cross-Section

FIG. 3 Electrical & Mechanical Stimulation Layout

FIG. 4 Sensor Suite and Signal Mapping

FIG. 5 Closed-Loop Control Diagram

FIG. 6 Vesicular Delivery Pathways

FIG. 7 Xylem Differentiation Stimulus Timeline

FIG. 8 Phloem Differentiation Stimulus Timeline

FIG. 9 Regenerative Wood Material Panel

### REFERENCE NUMERALS IN THE DRAWINGS

100 System housing/base unit
101 Tissue chamber block
102 Left electrode block
103 Right electrode block
104 Microfluidic tubing connection
105 Sensor bundle connection leaving chamber
106 Controller/processing unit
107 Actuator driver module
108 External user interface or computer (Host/UI)
109 Tissue/membrane strip
110 Flexible support frame
111 Left electrical contact pad
112 Right electrical contact pad
113 Mechanical actuator
114 Direction of stretch
115 Electrical drive source
120 Tissue region
121 Impedance sensor block
122 Strain sensor block
123 Optical sensor block
124 Ionic sensor block
125 Combined sensor bus
126 Controller block
130 Tissue/chamber block
131 Sensor block
132 Controller block
133 Actuator block
134 Feedback arrow lines
135 Stimulus arrow lines
140 Vesicle reservoir (e.g., syringe or container)
141 Microfluidic inlet line
142 Hydrogel patch attached near tissue
143 Tissue region
144 Vesicle flow
145 Immobilized vesicle layer
150 Time axis (horizontal)
151 Stimulus amplitude axis (vertical)
152 DC field curve
153 Shear profile curve
154 Mechanical strain pulses
160 Time axis
161 Stimulus amplitude axis
162 AC microcurrent envelope
163 Far-red light pulses
164 Reduced shear profile
170 Wood/composite panel
171 Embedded sensor zone
172 Embedded actuator/heater trace
173 Vascularized region
174 External connector pad or port
175 Direction of regeneration
201 Upper chamber wall/gasket
202 Lower chamber wall/gasket
203 Tissue region
204 Inlet channel
205 Outlet channel
206 Left electrode surface
207 Right electrode surface

### DETAILED DESCRIPTION

### Living-Physics Measurement And Control System

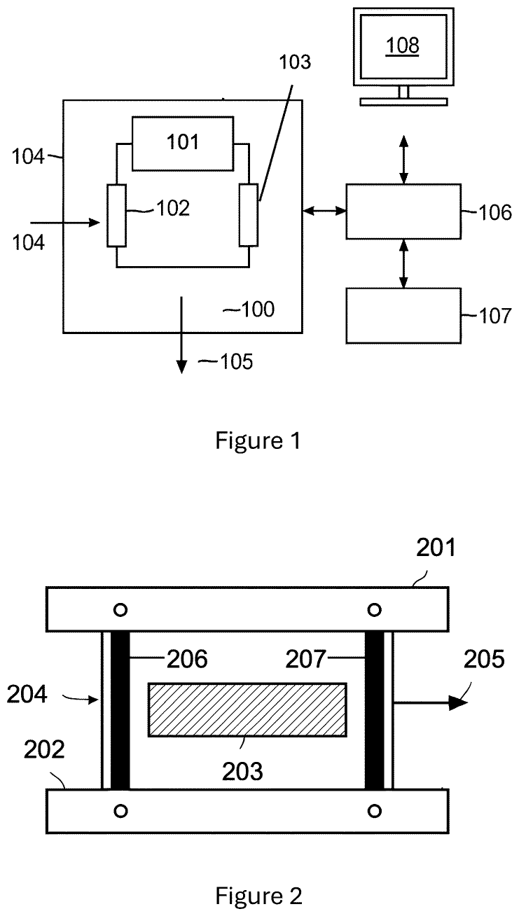

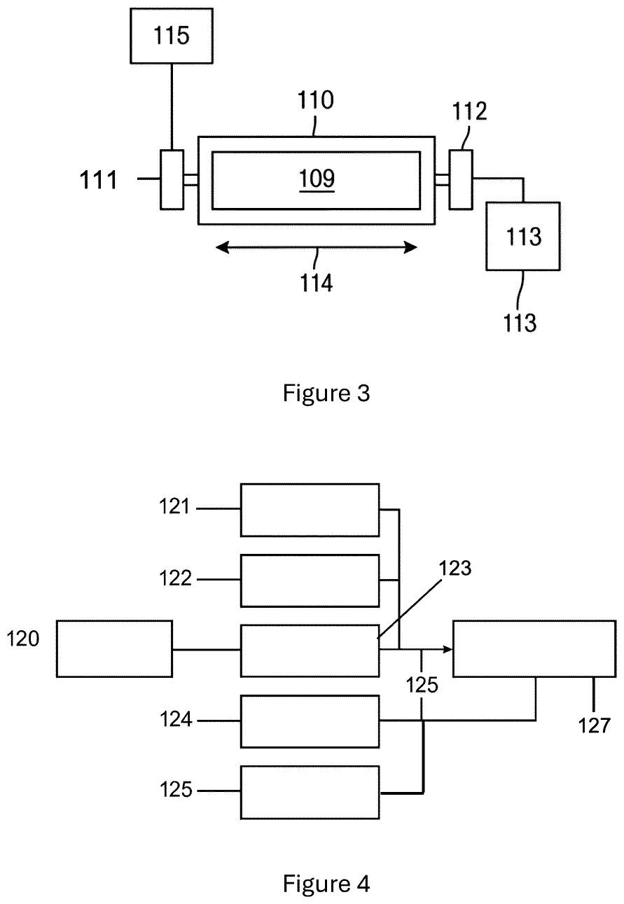

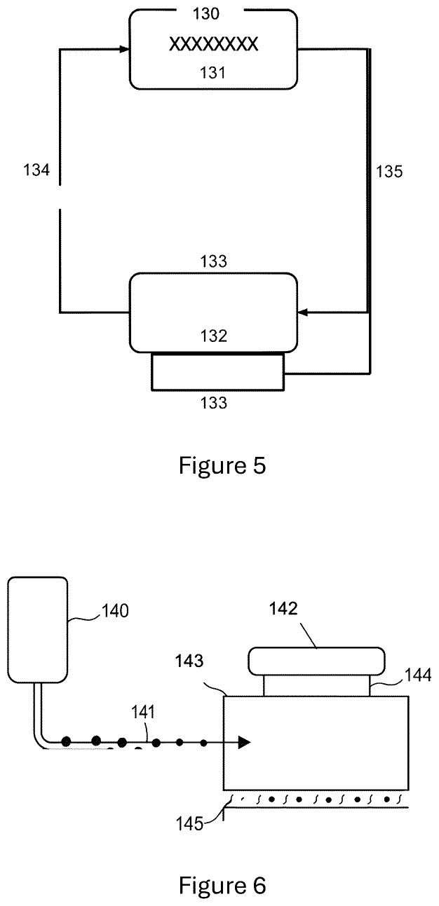

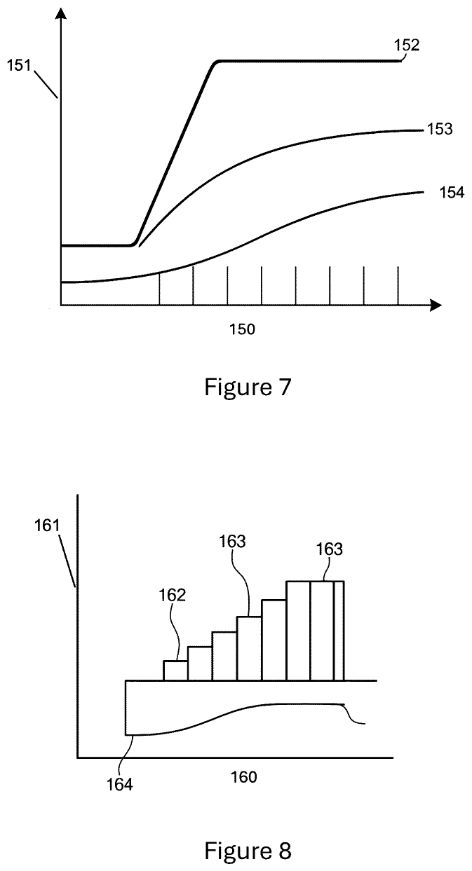

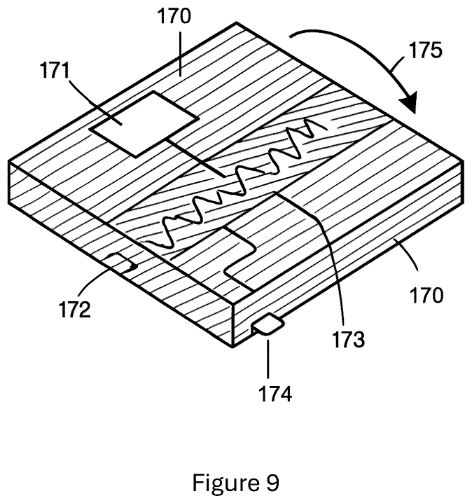

As shown in FIG. 1, the living-physics measurement and control system 100 can integrate a sensor array 105, an analog front-end, a controller module 106 with a firmware and signal-processing unit, a feedback actuator network 107, a data storage and communication interface, and a power supply, configured for low-voltage operation (e.g., 3.3-12 V DC) and scalable deployment. Generally, the controller module 106 can sample multimodal channels at rates between 1 Hz and 10 kHz with channel synchronization within ±1 ms and can compute order metrics including magnitude-squared coherence, entropy production rate σ, information flux, and a composite coherence-per-joule CPJ from timestamp-synchronized frames.

In one implementation, the feedback actuator network 107 can apply thermal, mechanical, ionic, and optical stimuli under proportional-integral-derivative and/or event-driven policies while the data storage and communication interface can log and communicate records for on-device and/or external analysis under low-power and/or high-throughput FPGA configuration and in distributed multi-node mesh variants. Therefore, the living-physics measurement and control system 100 provides integrated real-time instrumentation, quantitative links among coherence, entropy production, and information flow, and adaptive feedback to modulate energy routing in open systems, thereby enabling engineering of self-organizing materials with regenerative stability and addressing the identified technical challenges.

### Sensor Array

As shown in FIG. 1, the sensor array 105 can implement modular, synchronously sampled transducers configured to acquire physical signals from a target material and/or environment, with continuous and/or event-driven sampling at a target rate (e.g., 1 Hz to 10 kHz) and inter-channel drift maintained within ±1 ms over a 24-hour interval via a common reference clock.

In one implementation, the sensor array 105 includes thermal sensors (e.g., thermistors or thermocouples with ±0.1° C. resolution), mechanical/vibration sensors (e.g., piezoelectric films or MEMS accelerometers with 10-50 mV/g sensitivity), electrical/ionic sensors (e.g., Ag/AgCl or Pt electrode pairs measuring ±500 mV potentials and 10⁻³ to 10 S/m conductance), and optical sensors 123 (e.g., photodiodes with spectral filters covering 450-700 nm), with spatial arrangement and channel count selected to match monitored system heterogeneity and scale. Additionally, the sensor array 105 interfaces with an analog front-end for signal conditioning and digitization and communicates timestamp-synchronized data frames to a controller module 106 and a firmware and signal-processing unit to enable computation of magnitude-squared coherence, mutual information, entropy production rate, and composite coherence-per-joule metrics. Therefore, the sensor array 105 supplies an integrated, real-time multimodal sensor data stream that enables quantitative linking of coherence, entropy production, and information flow and supports closed-loop feedback to modulate energy flow and maintain functional order in open systems.

### Firmware & Signal-processing Unit

As shown in FIG. 1, the firmware and signal-processing unit can interface with the analog front-end and the clock and synchronization circuit to ingest a timestamp-synchronized sensor data frame and to execute load calibration coefficients and compute order metrics. Generally, the firmware and signal-processing unit can implement a task scheduler that processes a multimodal sensor data stream with a timing alignment maintained within a drift of approximately ±1 ms over an interval (e.g., over 24 hours). In one embodiment, the firmware and signal-processing unit can operate on a microcontroller, a microcomputer, and/or an FPGA to support real-time operation at a sampling rate (e.g., between 1 Hz and 10 kHz). Therefore, the firmware and signal-processing unit provides integrated real-time instrumentation that addresses a lack of synchronized measurement of energetic organization in open systems.

Generally, the firmware and signal-processing unit can calculate coherence by performing discrete Fourier transforms over sliding windows and by evaluating cross-spectral density to produce an averaged coherence index (c_avg). More specifically, the firmware and signal-processing unit can calculate entropy production by analyzing heat and temperature channels to produce an entropy production rate (σ) according to a thermodynamic relation such as over an interval (e.g., between 0.1 s and 60 s). In one implementation, the firmware and signal-processing unit can compute an information flux metric (IFI/MI/TE) using mutual-information estimators and can combine the averaged coherence index (c_avg) with an energy consumption log to calculate a composite coherence-per-joule metric (CPJ). Thus, the firmware and signal-processing unit generates quantitative metrics that link coherence, entropy production, and information flow.

Additionally, the firmware and signal-processing unit can store a stored calibration coefficient dataset and a baseline metrics record in non-volatile memory to enable adaptive thresholding and update baseline or learning parameters. In one implementation, the firmware and signal-processing unit can execute event-driven and/or continuous control logic to provide an actuator command set based on a computed energetic organization assessment, with configurable window lengths, estimator choices, and control gains. In another implementation, the firmware and signal-processing unit can adopt a low-power controller configuration on a duty-cycled microcontroller or a high-throughput FPGA configuration for dense channel counts, and can participate in a distributed multi-node mesh for spatially extended systems. Therefore, the firmware and signal-processing unit enables dynamic feedback to modulate energy flow and to preserve functional order while supporting adaptive, self-organizing operation.

### Evaluate Energetic Organization State

As shown in FIG. 1, the controller module 106 can evaluate the energetic organization state by ingesting the computed order metrics and the baseline metrics record to generate an energetic organization assessment. Generally, the controller module 106 can compare a composite coherence-per-joule metric, an entropy production rate, and an information flux metric against predetermined thresholds and/or adaptive statistical envelopes derived from baseline or historical data, and the controller module 106 can calculate deviations with optional weighting to yield a composite score. More specifically, the controller module 106 can perform time-series analysis using rolling windows (e.g., between 0.1 and 100 seconds) and update rates (e.g., between 1 and 1000 Hz) to detect persistent excursions and/or multi-scale trends, and the controller module 106 can apply hysteresis or confidence gating to stabilize graded indicators such as stable, approaching disorder, and disordered. In one implementation, the controller module 106 can execute the evaluation in firmware, software, or dedicated logic to meet an end-to-end latency target (e.g., between 0.5 and 50 ms), and the controller module 106 can output the energetic organization assessment continuously and/or at discrete control intervals to inform subsequent feedback actions.

### Execute Feedback Control

Generally, the controller module 106 can execute feedback control by generating an actuator command set from computed order metrics and an energetic organization assessment, where the firmware and signal-processing unit can compute deviations of a composite coherence-per-joule metric, an averaged coherence index, and/or an entropy production rate from target values and then synthesize control actions. More specifically, the controller module 106 can select control actions according to a control algorithm, such as proportional-integral-derivative regulation, model-predictive optimization, and/or reinforcement learning, and can update actuator setpoints at a fixed or event-driven rate (e.g., between 1 and 1000 Hz). In one implementation, the controller module 106 can command the feedback actuator network 107 to adjust thermal actuators 172 in watts or duty cycle, mechanical actuators 154 in phase/amplitude/frequency or RPM, ionic pumps 152 in microamperes to milliamperes, and/or optical emitters in irradiance or duty cycle, including phase-aligned drives for coherence restoration across nodes. Thus, the controller module 106 and the feedback actuator network 107 can close the loop that dynamically modulates energy and information flow, which addresses the absence of feedback mechanisms for preserving functional order in open systems and supports maintenance of an optimized energetic organization state.

### Update Baseline or Learning Parameters

Generally, the firmware & signal-processing unit can update baseline or learning parameters by incorporating computed order metrics into rolling statistics, such as exponentially weighted moving averages of the averaged coherence index, the entropy production rate, and the information flux metric, and can write an updated baseline metrics record to the data storage & communication interface 174. More specifically, the firmware & signal-processing unit can adjust adaptive thresholds for event detection and actuator setpoint priors, and can update weights and/or hyperparameters of embedded models, such as neural networks, regression models, or clustering algorithms, using accumulated multimodal sensor data. Additionally, the firmware & signal-processing unit can schedule the update according to a fixed time interval, a control-cycle count, or a detected statistical shift, can validate candidate updates via cross-validation and outlier rejection, and can commit revised coefficients to non-volatile memory for persistence across power cycles. In one implementation, the controller module 106 can select algorithms suited to a low-power controller configuration, a high-throughput FPGA configuration, or a distributed multi-node mesh, and the clock & synchronization circuit can timestamp parameter versions to enable rollback and audit of learning events.

### Wait Until Next Cycle

As shown in FIG. 1, the power management subsystem can execute the wait until next cycle step to regulate the temporal cadence of the coherence and entropy feedback control method. Generally, the clock and synchronization circuit can enforce a pause according to a fixed duration (e.g., between 10 ms and 10 s), a variable duration derived from a state estimator of the composite coherence-per-joule metric and/or entropy production rate, or an event-driven trigger such as a hardware interrupt, an external synchronization signal, or a completion flag from the feedback actuator network 107. In one implementation, the power management subsystem can enter a reduced-power or idle operating state during the pause while maintaining a timer peripheral and/or a software scheduler to preserve wake accuracy. Then, the controller module 106 can resume sensor acquisition, order-metric computation, and actuator command execution upon satisfaction of the configured wait condition.

In some embodiments, the present invention can be farther applied to a class of regenerative vascular materials using associated control systems capable of directing, stabilizing, or restoring plant-derived or plant-mimetic tissues through coordinated electrical, mechanical, thermal, optical, and biochemical stimuli. Bio-derived vesicular sensing elements are delivered in free form or immobilized formats to modulate the differentiation environment, while multimodal sensors (impedance, ionic, optical, thermal, and mechanical) compute order-disorder metrics that drive closed-loop actuation. The methods enable xylem, phloem, hybrid, or novel vascular architectures to form within natural or engineered wood-like substrates In one embodiment, the described architecture interfaces with a living or bio-ionic substrate that exhibits endogenous electrical and chemical activity. Sensors measure spontaneous potential differences and vibrational coherence within the material; actuators introduce gentle stimuli (heat <2° C. amplitude, micro-current <100 μA) to sustain or restore ordered patterns. Feedback improves stability of the biological interface without specifying molecular composition, demonstrating applicability to regenerative, self-healing, or bio-communicate.

In another embodiment, post-processing software generates coherence maps and entropy-flow diagrams showing directional energy routing. Statistical packages compute long-term averages and correlations to identify emergent self-organization. Data files use open CSV or HDF5 formats to maintain reproducibility.

In some embodiments, the thermal sensors are digital thermometers or thermocouples with ±0.1° C. resolution arranged along a temperature gradient. FIG. 2 illustrates one configuration in an entropy-routing laminate 200, where emissive surface 210 and conductive layer 220 sandwich an insulating core 230 containing sensors 212 and microchannels 240.

In some embodiments, the mechanical sensors are piezo-film or MEMS accelerometers detect strain and vibration. Signals (10-50 mV g⁻¹) are buffered by op-amps 122 before digitization.

In some embodiments, the electrical/ionic sensors are paired inert electrodes (Ag/AgCl or Pt) measure potentials up to ±500 mV and conductance between 10⁻³-10 S m⁻¹. AC excitation (≤100 mV rms at 1 kHz) ensures non-perturbative measurements.

In some embodiments, the optical sensors are photodiodes (BPW34) with optional spectral filters (450-700 nm) record luminescence or light scattering related to entropy production.

In some embodiments the signal processing and computation coherence calculation are achieved when the firmware performs of a discrete Fourier transform on each sensor signal x_i(t) to compute the magnitude-squared coherence. Entropy production rate is calculated σ(t)=\dot{Q}_out/T_env−\dot{Q}_in/T_sys. The mutual information I(A; B)=Σp(a, b)log₂[p(a, b)/(p(a)p(b))]. Composite Metric is compute as Coherence-per-Joule (CPJ): CPJ=C_avg/E_in. The control Logic (Algorithm): Start→Acquire signals→Compute C_avg, σ, CPJ→If |ΔC_avg|>threshold or σ>limit→Adjust actuators (PID loops for heater, fan, piezo)→Log data→Repeat.

In some embodiments, the disclosed measurements and control system provides the foundation for a dual-read biosensing indicator for detecting freshness, bacterial spoilage, and environmental stress in packaged food systems. Such device may employ a headspace-accessible hydrogel embedded with bio-derived extracellular-vesicle (EV)-based sensing elements that exhibit visible color and fluorescence changes in response to volatile organic compounds (VOCs), pH drift, moisture, or bacterial metabolites. A printed color reference enables naked-eye reading, while a smartphone camera +flash captures a fluorescence ratio. A freshness scoring algorithm fuses hue change (ΔHue), fluorescence ratio (G/R), and time-temperature exposure to yield an objective Freshness Index. An activation peel may initiate headspace exposure and time-stamp monitoring. The system may reduce waste, enables consumer trust, and supports \dot{Q}A across cold-chain logistics.

In some embodiments, the dual-read headspace biosensor is presented as a thin label (“freshness dot”) comprising: (i) a gas-permeable micro-cavity; (ii) an EV-functionalized hydrogel disc engineered to modulate optical properties in response to spoilage markers; (iii) a printed color reference for visual normalization; and (iv) a smartphone-read fluorescence channel. The system can be passive (human/phone read; no electronics) or integrated with semi-passive NFC (optional future variant). A Freshness Index (0-100) is computed from ΔHue, G/R, and time-temperature history, optionally applying penalties for abuse or humidity artifacts.

In some embodiments, the disclosed measurement and control system provide the foundation for a thin, semi-passive biosensor insert for detecting freshness, bacterial spoilage, and environmental stress in packaged foods. The system operates without batteries, harvesting power from near-field communication (NFC) to activate a flexible sensing tag. The insert comprises a bio-derived vesicular sensing layer responsive to volatile organic compounds (VOCs), moisture, and temperature, coupled with dual-modality transduction, optical and impedance. During an NFC interrogation, the device emits a controlled light pulse and measures corresponding optical scatter, fluorescence, and impedance changes of the sensing matrix. Processed results are transmitted to a mobile device, which computes freshness index based on signal normalization and environmental context.

In some embodiments, an NFC field is present, energy is harvested to power the circuit. A short optical pulse (typically 530 nm) excites the sensing layer. The photodiode measures reflected or emitted light intensity; concurrently, a low-frequency excitation (≈1 kHz) is applied across the impedance electrodes. The resulting signals reflect biochemical interactions between the sensing matrix and the package headspace environment. The controller samples and digitizes these signals, optionally averages multiple readings, and transmits compact numerical data (e.g., optical ratio, impedance magnitude, and temperature) to the reader device. A software application interprets these data as a freshness index.

In some embodiments, a freshness index may be calculated by combining normalized optical, impedance, and contextual parameters according to stored calibration coefficients. Calibration curves can be established for specific food categories such as seafood, poultry, or produce, using controlled spoilage studies. The algorithm may also apply correction factors for temperature-abuse events, detected from integrated sensor history.

In some embodiments, the biosensor insert can be manufactured using roll-to-roll flex printing including: conductive traces and antenna printed on PET or Kapton film, surface-mount placement of the NFC system-on-chip and sensors, hydrogel deposition or placement of pre-cast discs, lamination of the gas-permeable window and sealing adhesive. In some embodiments, all materials are selected to comply with food-contact or indirect-contact regulations (RoHS, REACH, FCN).

In some embodiments, a semi-passive biosensor insert for detecting freshness and spoilage in packaged foods, comprising: (a) a gas-permeable cavity; (b) a sensing matrix containing bio-derived vesicular sensing elements; (c) optical transduction components including a light source and photodetector; (d) impedance electrodes for dielectric measurement; and (e) an NFC-powered control circuit configured to activate the sensing components and transmit digital data to a reader device. The NFC field both powers the sensing circuit and provides bidirectional communication with a mobile device. The sensing matrix exhibits optical and impedance changes upon exposure to volatile amines, CO2, or other biochemical spoilage markers. The semi-passive biosensor insert, further comprising at least one environmental sensor selected from temperature, humidity, or VOC sensors. The sensing results are combined with contextual environmental data to compute a freshness index. The sensing layer is a hydrogel or polymer matrix that is food-safe and mechanically flexible. The antenna and circuitry are fabricated on a flexible substrate suitable for roll-to-roll manufacturing. The NFC event time-stamps the activation of the sensor upon first read. The optical response and impedance response are processed to yield a quantitative freshness score transmitted in digital format. In some embodiments, the biosensor operates without a battery or external wiring.

In some embodiments, the disclosed provide a platform for directing plant vascular differentiation, specifically xylem and phloem, by combining controlled electric fields, microcurrents, cyclic mechanical strain, microfluidic shear, light spectra, and micro-thermal pulses with a bio-derived vesicular sensing layer (vesicular biomolecules formulated as extracellular vesicle-like particles).

In some embodiments, the closed-loop controller measures multimodal signals (electrical impedance, micro-strain, ionic potentials, optical scattering) and dynamically adjusts stimuli to bias lineage outcomes. In some embodiments, the system increases coherence in measurable biophysical readouts and reduces drift, correlating with lumen formation, lignification (xylem) or sieve-element specification and callose patterning (phloem).

In some embodiments, a microphysiology bioreactor containing plant cells/tissues (callus, cambium ribbons, or organoids) exposed to programmable electrical (DC/AC) fields, mechanical stretch, microfluidic shear, light spectra, and micro-thermal pulses. A bio-derived vesicular sensing layer (vesicular biomolecules; EV-like) delivered in free solution or immobilized in slow-release hydrogel micro-patches to modulate signaling at tissue interfaces. An integrated sensor suite (electrical impedance spectroscopy, ionic microelectrodes, piezo micro-strain, optical imaging/OCT, micro-thermistors) feeding a controller that computes order/drift metrics and adapts stimuli in real time.

In some embodiments, methods and parameter ranges that bias xylem (lignified tracheary elements) versus phloem (sieve elements/companion cells) differentiation, with quantitative acceptance criteria and validation assays (marker panels, transport tests).

In some embodiments, the system enables deterministic programming of vascular architectures in plant tissues and wood-like composites and supports regenerative materials manufacturing.

In some embodiments, the disclosed may be implemented in a system for directing plant vascular tissue differentiation comprising: a tissue chamber; actuators configured to deliver electric fields and currents, cyclic mechanical strain, microfluidic shear, light spectra, and thermal pulses; sensors configured to measure at least electrical impedance and mechanical strain; and a controller programmed to adjust said actuators in response to sensor signals to bias differentiation toward xylem or phloem.

The system may further comprising a bio-derived vesicular sensing layer delivered in solution or immobilized proximate to the tissue to modulate signaling during differentiation, a controller applies DC fields between 50 and 300 mV/mm and AC currents between 10 and 100 μA/cm² at 100-1000 Hz, a controller applies cyclic mechanical strain between 0.5% and 2% at 0.1-1 Hz and shear stress 0.1-2 dyn/cm², a xylem-biased programs comprise reduced auxin, elevated cytokinin, and increased shear, and phloem-biased programs comprise elevated cytokinin, far-red light pulses, and reduced shear.

In some embodiments, the sensors include at least one of: ionic microelectrodes, optical scatter or OCT imaging, and micro-thermistors, and wherein sensor outputs are used to compute order/drift metrics for feedback control.

In some embodiments, a method of producing xylem-biased tissue comprising exposing plant cells to the system with DC field, cyclic strain, and micro-shear within the ranges recited, and delivering a bio-derived vesicular sensing layer according to a multi-day schedule.

In some embodiments, a method of producing phloem-biased tissue comprising exposing plant cells to AC microcurrents, reduced strain and shear, and far-red light pulses, while delivering a bio-derived vesicular sensing layer according to a multi-day schedule. In some embodiments, the differentiation is confirmed by marker expression (VND6/7, NST1/2, CesA4/7/8 for xylem; APL, SUC2, SWEET11/12, NAC45/86 for phloem) and functional transport assays (hydraulic conductivity or sugar translocation).

In certain embodiments, the disclosed provides a biophysical-biological control system designed to direct, stabilize, or restore vascular-like differentiation within plant-derived or plant-mimetic tissues. The system integrates multiple classes of physical stimuli, including controlled electrical fields ranging from approximately 10 to 400 mV/mm, microcurrents between about 1 and 200μA/cm² delivered at frequencies from 100 to 1,000 Hz, cyclic mechanical strain applied at amplitudes of about 0.1% to 5% and frequencies between 0.01 and 2 Hz, and microfluidic shear stress applied at levels between approximately 0.05 and 5 dyn/cm². Additional stimuli may include micro-thermal modulation through brief temperature elevations of roughly 1-2° C., as well as programmable light exposure involving defined red-to-blue ratios and intermittent far-red spectral pulses. These stimuli operate individually or in combination to establish a dynamic microenvironment capable of influencing morphogenetic pathways.

In further embodiments, the disclosed provides a multimodal sensor suite for monitoring the biological state of the tissue. This suite may incorporate sensors capable of electrical impedance spectroscopy over frequencies spanning 1 Hz to 1 MHz, piezoelectric micro-strain sensors capable of detecting subtle structural changes such as wall thickening or tissue stiffening, ionic potential electrodes that monitor ions including but not limited to potassium and calcium, optical sensors including optical coherence tomography modules that visualize lumenization and tissue alignment, and micro-thermistors that track local temperature stability. Collectively, these sensors capture continuous biophysical and biochemical information from the tissue.

In additional embodiments, the disclosed features a closed-loop controller configured to process sensor inputs into actionable control commands. The controller may compute metrics such as coherence between electrical and mechanical signals, drift and time-constant stability, coupling coefficients reflecting cross-domain predictiveness, and energy-normalized order metrics that collectively quantify the directional trajectory of differentiation. Based on these metrics, the controller adjusts one or more of the applied stimuli to maintain or restore a desired morphogenetic state. This closed-loop logic enables active morphogenetic control rather than passive culturing.

In yet further embodiments, the disclosed provides bio-derived vesicular sensing elements comprising extracellular vesicle-like biomolecular carriers derived from plant, algal, fungal, or synthetic biological sources. These vesicles may be delivered to the tissue in suspension at concentrations ranging from approximately 10⁸ to 10¹² vesicles per milliliter, or alternatively incorporated into slow-release hydrogels or immobilized surface films adjacent to the tissue. These elements modulate local signaling conditions without requiring disclosure of their specific internal cargo.

In still further embodiments, the disclosed provides regenerative vascular materials, including panels, laminates, or engineered scaffolds, that incorporate one or more of the systems described above. Such materials are capable of generating vascular-like architectures within the substrate, restoring structural or transport functionality following mechanical or environmental damage, and adapting to environmental disturbances through active feedback-controlled actuation.

As used herein, the term “vascular-like differentiation” refers to any structural or functional tissue formation resembling xylem, phloem, mixed xylem-phloem domains, or any novel form of transport-competent architecture not occurring in nature but produced by the systems and methods described in this specification.

As used herein, the term “bio-derived vesicular sensing elements” refers to extracellular-vesicle-like biomolecular structures derived from plant, algal, fungal, or synthetic sources. These vesicles may contain nucleic acids, proteins, lipids, metabolites, or other bioactive cargo, but their functional role for the present invention relates to modulating microenvironmental signaling conditions irrespective of specific cargo identity.

As used herein, the term “regenerative vascular material” refers to any plant-derived or plant-mimetic substrate that is capable of restoring, repairing, or otherwise improving one or more structural or functional properties—including mechanical continuity, hydraulic transport, carbohydrate translocation, or tissue alignment—through active morphogenetic control.

In one embodiment, the invention provides a tissue chamber constructed from materials such as PMMA, COC, glass, or ceramic. The chamber may include a central well with dimensions ranging from approximately 5 to 20 mm in diameter and a depth between roughly 0.3 and 1.0 mm. The chamber may include alignment guides to position callus tissue, cambium ribbons, or engineered scaffolds, and may incorporate an imaging window suitable for optical scatter measurements or OCT acquisition.

In additional embodiments, the invention incorporates multiple classes of actuators. Electrical actuation may involve direct-current fields between about 10 and 400 mV/mm or alternating-current microcurrents between about 1 and 200 μA/cm² at frequencies from 100 to 1,000 Hz. These may be delivered through Ag/AgCl or carbon electrodes, optionally stabilized by agar salt bridges. Mechanical actuation may deliver cyclic strain in the range of approximately 0.1% to 5% at frequencies between 0.01 and 2 Hz using servomotor, flexure, or voice-coil mechanisms. Shear stress may be introduced through microfluidic channels delivering forces between roughly 0.05 and 5 dyn/cm². Thermal actuation may involve brief temperature increases of 1-2° C. for durations ranging from 1 to 5 minutes. Optical actuation may include programmable red-blue spectral ratios and far-red pulses applied intermittently, for example 5-10 minutes per day.

In another embodiment, the sensor suite collects multimodal data describing the morphogenetic state of the tissue. Electrical impedance spectroscopy sensors detect changes associated with lignification and lumen formation. Piezoelectric micro-strain sensors detect changes in stiffness or wall thickening. Ionic microelectrodes track dynamic membrane potentials and ion flux signatures. Optical or OCT detectors visualize tissue organization, including lumenization, orientation, and sieve-plate formation. Thermal sensors maintain a record of temperature uniformity to ensure reproducible control conditions.

In further embodiments, the closed-loop controller integrates these sensor outputs over time. Sensor readings may be acquired every 5 to 15 minutes, and the controller may compute metrics such as coherence, drift, coupling, and energy-normalized order. If coherence decreases or drift increases beyond a defined threshold, the controller may apply corrective electrical fields, mechanical strain bursts, adjusted shear levels, or modified optical cues. The controller may also alter the release or exposure schedule of vesicular sensing elements to maintain the system's developmental trajectory.

In one embodiment, the biological tissue may include plant callus, showcasing undifferentiated cellular states suitable for inducible organoid-like vascularization. In another embodiment, the tissue may comprise cambium ribbons cut to thicknesses ranging from 100 to 300 micrometers and aligned to the anticipated transport axis. In yet another embodiment, the substrate may be a plant-based scaffold such as decellularized wood or cellulose-derived composite requiring regeneration.

Embodiments of the disclosed may use basal culture media such as MS or B5 supplemented with sucrose concentrations between 2% and 3% and adjusted to pH levels between approximately 5.7 and 5.8. A xylem-biased formulation may include relatively lower auxin levels, elevated cytokinin concentrations, and traces of brassinolide. A phloem-biased formulation may contain higher cytokinin levels, reduced auxin levels, and far-red optical cues delivered through the actuator system.

Bio-derived vesicular sensing elements may be introduced to a tissue through free suspensions, through hydrogel patches composed of materials such as PEG-DA or alginate, or through patterned microdomains immobilized on the scaffold surface. Vesicle concentrations may range from approximately 10⁸ to 10¹² vesicles per milliliter.

In one embodiment, a method for xylem differentiation is provided. The method may begin with pre-biasing the tissue for approximately 48 to 72 hours, followed by the application of direct-current fields between 100 and 200 mV/mm for intervals of 5 to 15 minutes per hour. Shear stress may be applied continuously at levels between 0.2 and 0.6 dyn/cm². Cyclic mechanical strain may be applied twice per day at amplitudes of about 1% and frequencies of 0.2 Hz. Vesicular sensing elements may be supplied on day 0 and boosted on day 4. Successful differentiation may be confirmed through staining, gene expression assays (such as CesA4/7/8), or measurements demonstrating a hydraulic conductivity at least twice that of control tissue.

In another embodiment, a method for phloem differentiation is provided. The method may include exposing the tissue to cytokinin-rich medium, applying alternating-current microcurrents at frequencies around 500 Hz and currents between approximately 10 and 30μA/cm², reducing strain to below 0.5%, and limiting shear stress to below 0.1 dyn/cm². Far-red light pulses of approximately 10 minutes per day may be administered. Vesicles may be delivered initially on day 0 and again on day 6. Successful differentiation may be confirmed through markers such as APL, SUC2, or SWEET11/12, through callose staining, or through measurements indicating at least a 1.5-fold increase in sugar flux relative to control.

In further embodiments, hybrid or novel vascular structures may be produced by alternating between different stimulation modalities, including alternating-polarity direct-current fields, orthogonal mechanical strain patterns, and periodic vesicle microbursts.

In one embodiment, the disclosed provides regenerative vascular materials constructed from wood veneers, engineered panels, composite laminates, or other plant-derived substrates. These materials incorporate the previously described tissue chambers, sensors, actuators, vesicle delivery interfaces, or combinations thereof. The materials autonomously detect structural or functional damage, initiate corrective morphogenetic stimuli, increase transport capacity, restore mechanical continuity, and thereby slow or reverse environmental or aging-related degradation.

In an illustrative embodiment, a xylem-biased organoid was produced from callus tissue over a 10-day period, demonstrating lumenization and achieving a hydraulic conductivity more than twice that of control tissue. In another embodiment, a phloem-biased tissue sheet was generated using alternating-current microcurrents and far-red light pulses, resulting in dye-transport efficiency reaching approximately 165% of that observed in control samples. In yet another embodiment, hybrid vascular patterning was achieved using alternating-polarity electrical fields, producing banded xylem-phloem structures not typically found in natural tissues. In a final embodiment, a regenerative engineered wood panel incorporating embedded sensors and heaters recovered approximately 32% of its hydraulic transport function after experiencing drying-induced cracking.

It should be understood that numerous variations of the present invention are possible. Electrodes may be planar, three-dimensional, flexible, rigid, or patterned to produce localized fields. Mechanical strain may be applied uniaxially, biaxially, torsional, or using a combination of these modes. Shear stress may be steady, oscillatory, or modulated according to closed-loop controller output. Light delivery systems may incorporate ultraviolet, infrared, or fully tunable spectral arrays. Vesicular sensing elements may be substituted with synthetic vesicle mimics or other biological nanoparticles that perform equivalent signaling functions. Substrates may be derived from natural wood, engineered cellulose composites, recycled plant biomass, or fully synthetic constructs designed to emulate the mechanical and biological properties of plant tissue.

I claim:

1. A living-physics measurement and control system comprising:
a) a sensor array including thermal sensors, mechanical/vibration sensors, electrical/ionic sensors, and optical sensors;
b) a clock & synchronization circuit configured to maintain inter-channel drift within ±1 ms over a 24-hour period;
c) an analog front-end configured to condition and digitize signals from the sensor array;
d) a controller module executing firmware configured to:
i) compute magnitude-squared coherence, an entropy-production rate, an information-flux metric, and a composite coherence-per-joule (CPJ) metric from digitized, time-aligned sensor data; and
ii) generate an actuator command set responsive to the computed metrics;
e) a feedback actuator network including at least one thermal actuator and at least one mechanical actuator, the feedback actuator network coupled to receive the actuator command set; and
f) a data storage & communication interface configured to log the computed metrics and the actuator command set.

2. A coherence and entropy feedback control method executed by the system of claim 1, the method comprising:
a) acquiring multimodal sensor data stream from the sensor array of the system;
b) synchronizing channel timestamps to create a timestamp-synchronized sensor data frame;
c) computing order metrics including an averaged coherence index (C_avg), an entropy-production rate (σ), an information-flux metric (IFI), and a composite CPJ;
d) evaluating an energetic organization state by comparing at least one of the order metrics
with a baseline metrics record;
e) determining actuator adjustments when a threshold-violation event is detected;
f) applying actuator commands to at least one actuator in the feedback actuator network of the system;
g) updating the baseline metrics record after application of the actuator commands.

3. The system of claim 1, wherein the sensor array comprises:
a) time-synchronized sampling rates selectable between 1 Hz and 10 kHz;
b) a per-channel digital resolution of at least 16 bits;
c) spatially distributed placement configured to monitor a common tissue or material region.

4. The system of claim 1, wherein the controller module executing the firmware calculates the composite coherence-per-joule (CPJ) metric by:
d) integrating cumulative energy consumed from an energy consumption log;
e) averaging coherence over a defined bandwidth;
f) dividing the averaged coherence by the cumulative energy to yield the composite coherence-per-joule metric in J⁻¹.

5. The system of claim 1, wherein the feedback actuator network further comprises:
a) ionic pumps configured for electrophoretic ion transport below 100 μA;
b) optical emitters providing 450-700 nm illumination up to 50 mW per channel.

6. The system of claim 1, further comprising a power management subsystem configured to:
a) operate from a supply below 12 V DC;
b) gate power to individual sensor and actuator channels to maintain a total power draw under 5 W.

7. The system of claim 1, wherein the data storage and communication interface provides:
a) removable SD-card logging in CSV or HDF5 format;
b) wireless communication via Wi-Fi or Bluetooth Low Energy for remote dashboard visualization.

8. The system of claim 1, implemented in a low-power controller configuration, wherein:
a) the controller module comprises an ultra-low-power microcontroller; and
b) peripherals are duty-cycled to keep average system power below 250 mW.

9. The system of claim 1, implemented in a high-throughput FPGA configuration, wherein:
a) the controller module comprises a field-programmable gate array;
b) the system sustains an aggregate sampling rate above 1 MS/s;
c) the system achieves control latency below 1 ms while supporting more than 64 synchronous sensor channels.

10. The system of claim 1, deployed as a distributed multi-node mesh, further comprising:
a) a plurality of identical controller boards that share a deterministic real-time network with sub-microsecond synchronization; and
b) wherein order metrics are aggregated across square-meter to building-scale installations.

11. The method of claim 2, further comprising an initialization step that:
a) retrieves a stored calibration coefficient dataset for each sensor channel;
b) verifies sensor connectivity and actuator readiness;
c) establishes a calibrated system ready configuration.

12. The method of claim 2, further comprising training a baseline coherence profile by:
a) acquiring quiescent multimodal data with no actuation;
b) computing baseline C_avg, σ, IFI, and CPJ;
c) storing the values as a baseline metrics record.

13. The method of claim 2, wherein computing the information-flux metric includes:
a) estimating mutual information I(A; B)=Σp(a, b) log₂[p(a, b)/(p(a)p(b))] over rolling windows; and
b) optionally calculating transfer entropy for directional coupling.

14. The method of claim 2, wherein determining actuator adjustments comprises:
a) executing a proportional-integral-derivative (PID) control loop that minimizes deviation of CPJ from a target value; and
b) constraining actuator commands within predefined safety limits.

15. The method of claim 2, wherein the method further comprises generating an actuator command set, the actuator command set including:
a) pulse-width modulation duty cycles for thermal actuators;
b) phase-aligned drive waveforms for mechanical actuators;
c) current amplitude settings for ionic pumps.

16. The method of claim 2, further comprising logging and communicating data by:
a) recording raw sensor data, computed order metrics, and actuator commands to local storage; and
b) transmitting selected summaries to a cloud server for long-term analytics.

17. The method of claim 2, wherein updating the baseline metrics record utilizes:
a) an exponentially weighted moving average with a user-selectable decay factor; and
b) rejection of outliers beyond three standard deviations from historical means.

18. The method of claim 2, wherein the steps from acquiring multimodal sensor data stream through applying actuator commands are repeated at a control-loop cadence between 10 ms and 10 s.

19. The method of claim 2, carried out on the low-power controller configuration of claim 8 to enable battery-powered operation.

20. The method of claim 2, resulting in an optimized energetic organization state characterized by:
a) CPJ exceeding a predetermined threshold;
b) entropy-production rate σ at or below a target limit;
c) the optimized energetic organization state being sustained for at least a user-defined hold time.
